(19) United States
(12) Patent Application Publication
(10) Pub. No.: US 2023/0233097 A1
(43) Pub. Date: Jul. 27, 2023

(54) CUSTOMIZED PATIENT TRACKER FOR IMAGE GUIDED SURGERY

(71) Applicants: Acclarent, Inc., Irvine, CA (US); Biosense Webster (Israel) Ltd., Yokneam (IL)

(72) Inventors: Itzhak Fang, Irvine, CA (US); Babak Ebrahimi, Irvine, CA (US); Ehsan Shameli, Irvine, CA (US); Uriel Hod, Haifa (IL); Itamar Bustan, Zichron Ya'acov (IL); Jetmir Palushi, Irvine, CA (US)

(21) Appl. No.: 18/074,678

(22) Filed: Dec. 5, 2022

Related U.S. Application Data
(60) Provisional application No. 63/303,698, filed on Jan. 27, 2022.

(51) Int. Cl. A61B 5/06; A61B 34/20

(52) U.S. Cl. CPC A61B 5/062; A61B 34/20

(57) ABSTRACT

An apparatus includes a mounting portion and a sensor portion. The mounting portion is configured to fit over a nose of a preselected patient. The mounting portion includes a base and a pair of rigid nose pads fixedly coupled to the base. Each nose pad of the pair of nose pads includes a respective nose-gripping surface. The nose-gripping surfaces of the nose pads are configured to engage the nose of the preselected patient at respective predetermined locations along the nose. Each nose-gripping surface is sized and shaped to complement a corresponding unique structural feature of the nose of the preselected patient at the predetermined location. A sensor portion is fixedly attached to the mounting portion. The sensor portion includes a first sensor configured to generate a first signal corresponding to a position of the first sensor in three-dimensional space.

### PRIORITY

This application claims priority to U.S. Provisional Pat. App. No. 63/303,698, entitled “Customized Patient Tracker for Image Guided Surgery,” filed Jan. 27, 2022, the disclosure of which is incorporated by reference herein, in its entirety.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pub. No. 2011/0004057, entitled “Systems and Methods for Transnasal Dilation of Passageways in the Ear, Nose or Throat,” published Jan. 6, 2011, now abandoned, the disclosure of which is incorporated by reference herein. An example of such a system is the Relieva® Spin Balloon Sinuplasty™ System by Acclarent, Inc. of Irvine, Calif.

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) mounted thereon are used to perform the procedure while the sensors send data to the computer indicating the current position of each surgical instrument. The computer correlates the data it receives from the instrument-mounted sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. In this manner, the surgeon is able to know the precise position of each sensor-equipped instrument by viewing the video monitor even if the surgeon is unable to directly visualize the instrument itself at its current location within the body.

An example of an electromagnetic IGS systems that may be used in ENT and sinus surgery is the CARTO® 3 System by Biosense-Webster, Inc., of Irvine, Calif. When applied to functional endoscopic sinus surgery (FESS), balloon sinuplasty, and/or other ENT procedures, the use of IGS systems allows the surgeon to achieve more precise movement and positioning of the surgical instruments than can be achieved by viewing through an endoscope alone. As a result, IGS systems may be particularly useful during performance of FESS, balloon sinuplasty, and/or other ENT procedures where anatomical landmarks are not present or are difficult to visualize endoscopically.

One function that may be performed by an IGS system is obtaining a reference point that can be used to correlate various preoperatively obtained images with a patient's actual position during a procedure. This act may be referred to as patient registration. Patient registration is conventionally performed by using a positionally tracked instrument (e.g., a guidewire whose tip position may be detected in three-dimensional space) to trace the area of a patient that will be affected by the procedure. For example, in the case of a balloon sinuplasty or other ENT procedure, a positionally tracked guidewire or other tool may be used to trace or touch one or more positions on a patient's face. At each touch point, a positional tracking system will register that point in three-dimensional space and, using a number of registered points, determine the position of the affected area in three-dimensional space. Once the affected area is fully mapped or registered, it can be correlated with preoperative images in order to provide a seamless IGS experience across varying types of preoperative images during the performance of the procedure. Performing patient registration in this manner is both time consuming and error prone, due to the number of touch points required for some procedures and the relative inaccuracy of pressing a flexible guidewire tip against the non-rigid surface of a patient's face.

It may be desirable to provide features that further facilitate the use of an IGS navigation system and associated components in ENT procedures and other medical procedures. While several systems and methods have been made and used with respect to IGS and ENT surgery, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 depicts a schematic view of an exemplary sinus surgery navigation system being used on a patient seated in an exemplary medical procedure chair;

FIG. 2 depicts a front perspective view of an exemplary registration probe having a position sensing tip;

FIG. 3 depicts a front elevation view of a patient during a registration procedure using the registration probe of FIG. 2;

FIG. 4 depicts a front elevation view of an exemplary customized patient tracking assembly;

FIG. 5 depicts a rear perspective view of the patient tracking assembly of FIG. 4;

FIG. 6 depicts a side elevation view of a patient;

FIG. 7 depicts a front elevation view of the patient of FIG. 6 with the patient tracking assembly of FIG. 4 positioned over the patient's nose; and

FIG. 8 depicts a side elevation view of the patient of FIG. 6 with the patient tracking assembly of FIG. 4 positioned over the patient's nose.

### DETAILED DESCRIPTION

The inventors have conceived of novel technology that, for the purpose of illustration, is disclosed herein as applied in the context of image guided surgery. While the disclosed applications of the inventors' technology satisfy a long-felt but unmet need in the art of image guided surgery, it should be understood that the inventors' technology is not limited to being implemented in the precise manners set forth herein, but could be implemented in other manners without undue experimentation by those of ordinary skill in the art in light of this disclosure. Accordingly, the examples set forth herein should be understood as being illustrative only, and should not be treated as limiting.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

### I. Exemplary Image Guided Surgery Navigation System

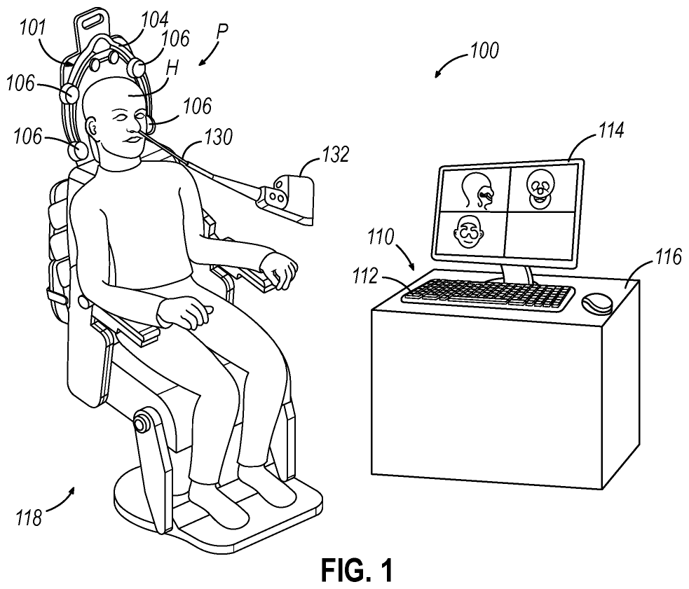

FIG. 1 shows an exemplary IGS navigation system (100) enabling an ENT procedure to be performed using image guidance. In some instances, IGS navigation system (100) is used during a procedure where a dilation instrument assembly (not shown) is used to dilate the ostium of a paranasal sinus; or to dilate some other anatomical passageway (e.g., within the ear, nose, or throat, etc.). In addition to or in lieu of having the components and operability described herein IGS navigation system (100) may be constructed and operable in accordance with at least some of the teachings of U.S. Pat. No. 8,702,626, entitled “Guidewires for Performing Image Guided Procedures,” issued Apr. 22, 2014, the disclosure of which is incorporated by reference herein; U.S. Pat. No. 8,320,711, entitled “Anatomical Modeling from a 3-D Image and a Surface Mapping,” issued Nov. 27, 2012, the disclosure of which is incorporated by reference herein; U.S. Pat. No. 7,720,521, entitled “Methods and Devices for Performing Procedures within the Ear, Nose, Throat and Paranasal Sinuses,” issued May 18, 2010, the disclosure of which is incorporated by reference herein; U.S. Pat. Pub. No. 2014/0364725, entitled “Systems and Methods for Performing Image Guided Procedures within the Ear, Nose, Throat and Paranasal Sinuses,” published Dec. 11, 2014, now abandoned, the disclosure of which is incorporated by reference herein; U.S. Pat. No. 10,362,965, entitled “System and Method to Map Structures of Nasal Cavity,” issued Jul. 30, 2019; and U.S. Pat. Pub. No. 2011/0060214, entitled “Systems and Methods for Performing Image Guided Procedures within the Ear, Nose, Throat and Paranasal Sinuses,” published Mar. 10, 2011, now abandoned, the disclosure of which is incorporated by reference herein.

IGS navigation system (100) of the present example comprises a field generator assembly (101), which comprises set of magnetic field generators (106) that are integrated into a horseshoe-shaped frame (104). Field generators (106) are operable to generate alternating magnetic fields of different frequencies around the head of the patient. Field generators (106) thereby enable tracking of the position of a navigation guidewire (130) that is inserted into the head of the patient. Various suitable components that may be used to form and drive field generators (106) will be apparent to those of ordinary skill in the art in view of the teachings herein.

In the present example, frame (104) is mounted to a chair (118), with the patient (P) being seated in the chair (118) such that frame (104) is located adjacent to the head (H) of the patient (P). By way of example only, chair (118) and/or field generator assembly (101) may be configured and operable in accordance with at least some of the teachings of U.S. Pat. No. 10,561,370, entitled “Apparatus to Secure Field Generating Device to Chair,” issued Feb. 18, 2020, the disclosure of which is incorporated by reference herein.

IGS navigation system (100) of the present example further comprises a processor (110), which controls field generators (106) and other elements of IGS navigation system (100). For instance, processor (110) is operable to drive field generators (106) to generate electromagnetic fields; and process signals from navigation guidewire (130) to determine the location of a sensor in navigation guidewire (130) within the head (H) of the patient (P). Processor (110) comprises a processing unit communicating with one or more memories. Processor (110) of the present example is mounted in a console (116), which comprises operating controls (112) that include a keypad and/or a pointing device such as a mouse or trackball. A physician uses operating controls (112) to interact with processor (110) while performing the surgical procedure.

A coupling unit (132) is secured to the proximal end of a navigation guidewire (130). Coupling unit (132) of this example is configured to provide wireless communication of data and other signals between console (116) and navigation guidewire (130). While coupling unit (132) of the present example couples with console (116) wirelessly, some other versions may provide wired coupling between coupling unit (132) and console (116). Various other suitable features and functionality that may be incorporated into coupling unit (132) will be apparent to those of ordinary skill in the art in view of the teachings herein.

Navigation guidewire (130) may be used in a dilation instrument (not shown). Navigation guidewire (130) includes a sensor (not shown) that is responsive to movement within the fields generated by field generators (106). In the present example, the sensor of navigation guidewire (130) comprises at least one coil at the distal end of navigation guidewire (130). When such a coil is positioned within an electromagnetic field generated by field generators (106), movement of the coil within that magnetic field may generate electrical current in the coil, and this electrical current may be communicated along the electrical conduit(s) in navigation guidewire (130) and further to processor (110) via coupling unit (132). This phenomenon may enable IGS navigation system (100) to determine the location of the distal end of navigation guidewire (130) within a three-dimensional space (i.e., within the head (H) of the patient (P)). To accomplish this, processor (110) executes an algorithm to calculate location coordinates of the distal end of navigation guidewire (130) from the position related signals of the coil(s) in navigation guidewire (130).

Processor (110) uses software stored in a memory of processor (110) to calibrate and operate system (100). Such operation includes driving field generators (106), processing data from navigation guidewire (130), processing data from operating controls (112), and driving display screen (114). Processor (110) is further operable to provide video in real time via display screen (114), showing the position of the distal end of navigation guidewire (130) in relation to a video camera image of the patient's head (H), a CT scan image of the patient's head (H), and/or a computer-generated three-dimensional model of the anatomy within and adjacent to the patient's nasal cavity. Display screen (114) may display such images simultaneously and/or superimposed on each other during the surgical procedure. Such displayed images may also include graphical representations of instruments that are inserted in the patient's head (H), such as navigation guidewire (130), such that the operator may view the virtual rendering of the instrument at its actual location in real time. By way of example only, display screen (114) may provide images in accordance with at least some of the teachings of U.S. Pat. No. 10,463,242, entitled “Guidewire Navigation for Sinuplasty,” issued Nov. 5, 2019, the disclosure of which is incorporated by reference herein. In the event that the operator is also using an endoscope, the endoscopic image may also be provided on display screen (114).

The images provided through display screen (114) may help guide the operator in maneuvering and otherwise manipulating instruments within the patient's head. When used in a dilation instrument assembly (not shown), navigation guidewire (130) may facilitate navigation of instrumentation of the dilation instrument assembly within the patient during performance of a procedure to dilate the ostium of a paranasal sinus; or to dilate some other anatomical passageway (e.g., within the ear, nose, or throat, etc.). It should also be understood that other components of the dilation instrument assembly may incorporate a sensor like the sensor of navigation guidewire (130), including but not limited to a dilation catheter (not shown).

### II. Exemplary Touch Instrument to Register and Calibrate Image Guided Surgery System

As noted above, a variety of guidewires may be used to perform registration and calibration in an IGS navigation system (100) by touching various points on the patient's face with the positionally tracked guidewire tip. Such guidewires may be rather flimsy or flexible by their very nature. This flexibility may make it difficult for an operator to grasp the guidewire by itself and manipulate the distal tip of the guidewire to contact registration points on the patient's head. For instance, the distal tip of the guidewire may tend to deflect in response to engagement with the patient's head, which may compromise the accuracy of the registration. It may therefore be desirable to at least temporarily provide rigidity to a guidewire during the process of registration and calibration in an IGS navigation system (100). Such added rigidity may make it easier for the operator to handle the guidewire, may prevent the distal tip of the guidewire from deflecting in response to engagement with the patient's head, and may ultimately provide a more accurate registration.

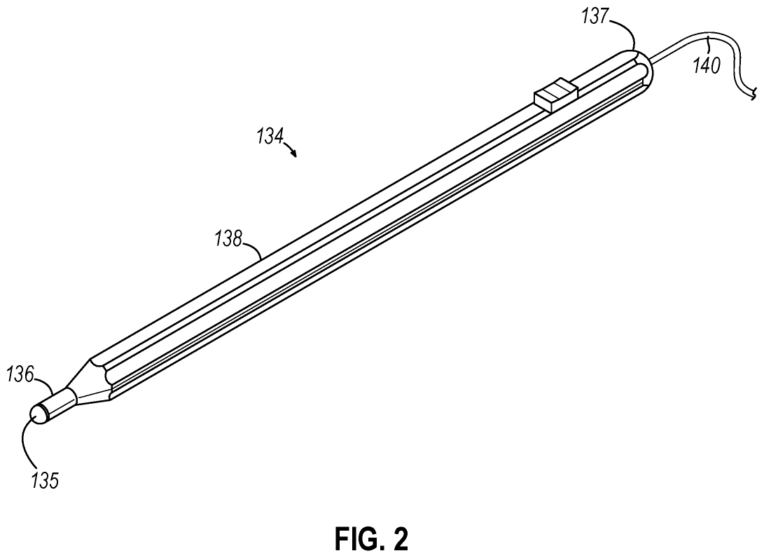

FIG. 2 show an exemplary touch-based registration and calibration instrument (134) that may be used to temporarily provide rigidity to an otherwise flimsy guidewire in order to register and calibrate an IGS navigation system such as IGS navigation system (100) described above. Calibration instrument (134) of this example comprises a rigid elongate body (138) having a distal end (136) and a proximal end (137). In some versions, elongate body (138) is formed of a transparent polycarbonate material. Distal end (136) includes a taper leading to a reduced diameter portion, which ultimately terminates in a rounded distal tip (135).

A guidewire (140) may be inserted into the rigid elongate body (138) so that an end of the guidewire (140) rests against the interior of the rounded distal tip (135). Since end of the guidewire (140) is positionally tracked, the rounded distal tip (135) may be used to touch a registration point on a patient's face, which will place the positionally tracked tip within close proximity of the registration point, separated only by the known width of a wall of the rigid elongate body (138). In this configuration, instrument (134) may be used to perform the registration and calibration process associated with IGS navigation system (100) by touching the rounded distal tip (135) to each registration point while providing another input to the system, such as interacting with a foot pedal or button, speaking a voice command, or another similar input, to cause the registration touch to be recorded. In some versions, calibration instrument (134) includes a contact sensor (not shown) that senses when the distal tip (135) contacts the face of the patient. In some such versions, the operator must press distal tip (135) against the face of the patient with enough force to overcome a threshold for the contact sensor to register the contact between distal tip (135) and the face of the patient.

It should also be understood that while distal tip (135) will be contacting registration points on the patient's head or face instead of the positionally tracked tip of the guidewire (140) contacting those registration points, the system may readily make the necessary adjustments in the registration and calibration algorithms in view of the fact that the width of the wall of the rigid elongate body (138) is fixed and known.

The calibration instrument (134) or probe of FIG. 2 may provide rigidity to the flexible guidewire (140) during the registration process, which can address one source of inaccuracy (e.g., flexing of the positionally tracked guidewire during the touch). However, it does not address other sources of inaccuracy, for example, that may be introduced due to the flexibility or pliability of flesh on a patient's head or face as the distal tip (135) is pressed against it during the registration process. Various points on a patient's face may depress several millimeters under the force of the calibration instrument (134), which can provide a significant inaccuracy in the context of an ENT or other surgical procedure.

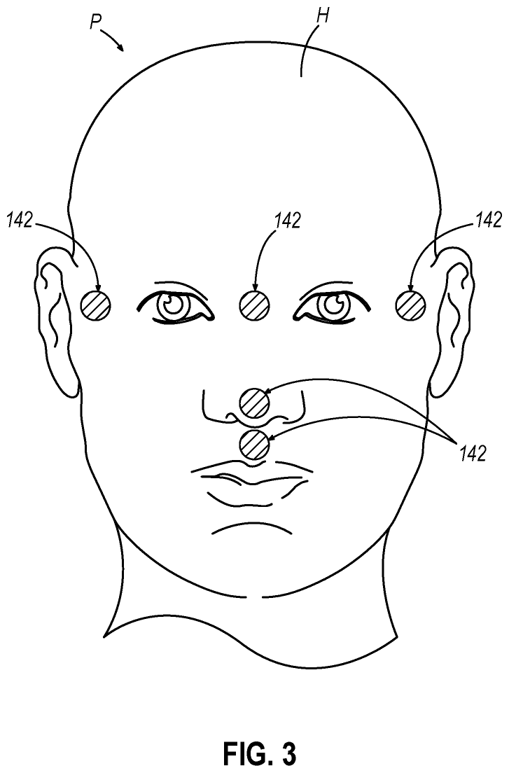

FIG. 3 shows a front elevation view of a patient (P) showing a patient registration procedure using the calibration instrument (134). The view of FIG. 3 might be rendered on a display of the IGS system (100) during a procedure, and may show one or more registration points (142) that must be calibrated or registered using the calibration instrument (134). The registration points may, for example, be shown in one color before they are registered with a touch of the calibration instrument (134), and may change to a different color or otherwise indicate calibration after a touch of the calibration instrument (134). In some versions, one or more lasers are used to project the registration points (142) on the face of the patient, such that the operator must engage the face of the patient (P) with distal tip (135) at each point illuminated by the laser(s).

In addition to the foregoing, calibration instrument (134) may be configured and operable in accordance with at least some of the teachings of U.S. Pat. No. 10,779,891, entitled “System and Method for Navigation of Surgical Instruments,” issued as on Sep. 22, 2020, the disclosure of which is incorporated by reference herein.

As can be seen from the example of FIG. 3, this registration may require five touches of the calibration instrument (134) at different points of the patient's face to be completed. In real world use, the number of registration points can be in the tens or even hundreds. In this context, it becomes apparent that it can be a very time-consuming process to identify, locate, and touch each required point.

### III. Exemplary Customized Patient Tracking Assembly for Image Guided Surgery

When field generators (106) are in fixed positions relative to chair (118) during exemplary use, rather than being in fixed positions relative to head (H) of patient (P), the frame of reference for IGS navigation system (100) (i.e., the electromagnetic field generated by field generators (106)) does not move with the head (H) of the patient (P). In some instances, a procedure may involve intentional or inadvertent movements by the patient (P) while situated in chair (118), such that the patient's head (H) may shift positions, location, and/or orientation in relation to frame (104). When a navigation guidewire (130) (or other instrument having a sensor compatible with IGS navigation system (100)) is disposed in the head (H) of the patient (P), IGS navigation system (100) may not be able to differentiate between (i) movement of navigation guidewire (130) relative to the head (H) of the patient and (ii) movement of the head (H) of the patient (P) with navigation guidewire (130) (e.g., when navigation guidewire (130) remains stationary relative to the head (H) of the patient (P) yet moves with the head (H) relative to frame (104)). Thus, by not securing field generators (106) relative to the head (H) of the patient (P), IGS navigation system (100) may provide inaccurate position data relative to the head (H) of the patient (P) when the head (H) of the patient (P) moves while navigation guidewire (130) is disposed in the head (H) of the patient (P). It may therefore be desirable for IGS navigation system (100) to include features and functionality to account for movement of the patient's head (H), to preserve the accuracy of IGS navigation system (100).

For instance, it may be beneficial to incorporate navigation system components, such as sensors, onto a patient's head (H) that are configured to generate signals indicating the real-time position (and movement) of the patient's head (H) within the field generated by field generators (106), and communicate such signals to IGS navigation system (100), such that movement of a patient's head (H) may be separately tracked and thereby accounted for by IGS navigation system (100). It may further be desirable for a sensor that is dedicated to tracking patient head (H) movement to be unobtrusive. Less intrusive means of attaching navigation system components onto a patient's head may be beneficial for ease in installation and enhanced comfort for the patient during the procedure.

The following description provides examples of a navigation system component in the form of a patient tracking device that is configured to cooperatively communicate with IGS navigation system (100) to improve accuracy in tracking the position of an instrument (e.g., navigation guidewire (130)) that is inserted into the patient's head (H). In particular, the patient tracking device is configured to generate signals indicating the real-time position (and movement) of the patient's head (H) within the field generated by field generators (106), such that the signals generated by a navigational instrument (e.g., navigation guidewire (130)) may be processed by processor (110) through an error correction algorithm, to effectively subtract-out patient head (H) movement, to thereby accurately determine the three-dimensional location of the navigational instrument within the head (H) of the patient (P). The patient tracking device described below may also provide registration of the patient (P) relative to IGS navigation system (100), such that the patient tracking device may obviate the need for using a separate registration instrument like calibration instrument (134).

It should be understood that the patient tracking devices described below may be readily incorporated into any of the various navigation systems (100) described above and in any of the various medical procedures described in the various references described herein. Other suitable ways in which the below-described patient tracking devices may be used will be apparent to those of ordinary skill in the art in view of the teachings herein.

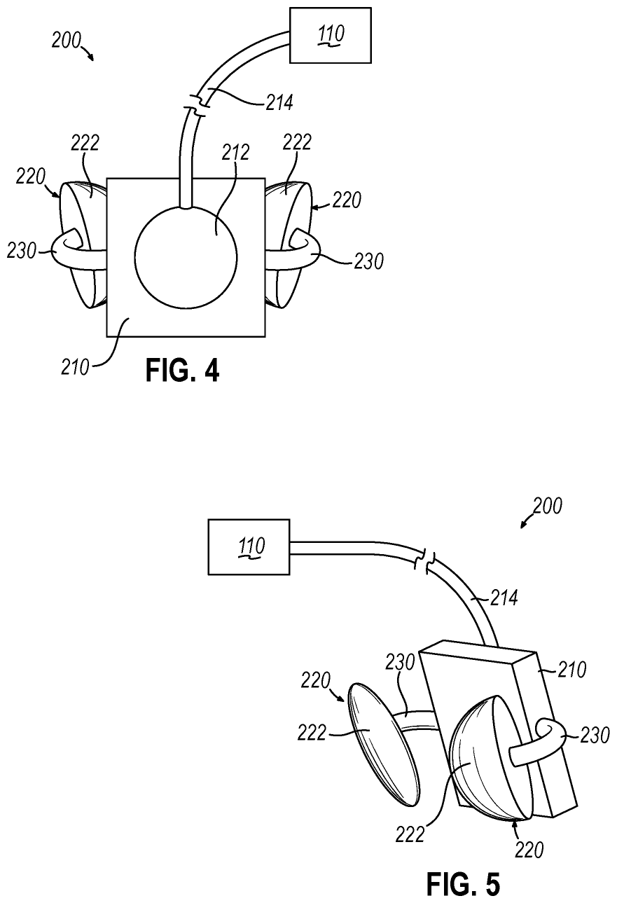

FIGS. 4-5 depict an exemplary customized patient tracking assembly (200) that may be readily incorporated into IGS navigation system (100) where field generators (106) are not fixed to the head (H) of the patient (P). As will be described in greater detail below, patient tracking assembly (200) includes a base (210) and a sensor portion (212). As will also be described in greater detail below, base (210) partially defines a mounting portion of patient tracking assembly (200) that is configured to attach to the head (H) of the patient (P) and to fixedly couple with sensor portion (212) such that sensor portion (212) is fixed relative to the head (H) of the patient (P) during exemplary use; while sensor portion (212) is configured to communicate with processor (110) in order to track the position of the head (H) of the patient (P) relative to field generators (106) during exemplary use.

In this regard, sensor portion (212) houses a position sensor (not shown) fixedly therein, which may be substantially similar to the sensor (not shown) from navigation guidewire (130) described above, with differences elaborated below. By way of example only, the sensor may comprise one or more coils of wire wrapped around one or more respective axes. The sensor may be responsive to positioning within alternating magnetic fields generated by field generators (106) during exemplary use, such that the sensor generates signals indicating the real-time position of the sensor within alternating magnetic fields generated by field generators (106). Signals generated by the sensor may be sent to processor (110), such as via a cable (214) extending proximally from sensor portion (212) to processor (110).

Because sensor portion (212) is fixedly attached to base (210) during exemplary use, and because base (210) is fixed to the head (H) of the patient (P) as described below, signals generated by the sensor of sensor portion (212) may be indicative of the real-time position (and movement) of the head (H). Therefore, signals generated by the sensor may allow processor (110) to determine the three-dimensional location and orientation of the head (H) of the patient (P) relative to the plurality of field generators (106) fixed to frame (104). Processor (110) may utilize this information, in conjunction with the location of navigation guidewire (130) (or any other suitable instrument) relative to field generators (106), such that processor (110) and display screen (114) may properly display the real-time location of navigation guidewire (130) (or any other suitable instrument) within the head (H) of the patient (P).

Cable (214) of the present version is configured to provide a conduit for communication between the sensor of sensor portion (212) and processor (110) during exemplary use. Therefore, cable (214) may directly connect with console (116) such that the sensor of sensor portion (212) is in wired communication with processor (110) via cable (214). Alternatively, cable (214) may connect the sensor of sensor portion (212) with a wireless communication device that is in wireless communication with console (116), similar to how coupling unit (132) establishes wireless communication between navigation guidewire (130) and console (116). Cable (214) may provide a conduit for communication between the sensor of sensor portion (212) and processor (110) through any suitable techniques known to one having ordinary skill in the art in view of the teachings herein. As another variation, sensor portion (212) may have an integral wireless transmitter that is operable to wirelessly communicate signals from sensor portion (212) to processor (110). Cable (214) may thus be omitted in some versions.

As mentioned above, base (210) is configured to fixedly couple with sensor portion (212). For example, base (210) may include a receptacle (not shown) for at least partially receiving sensor portion (212) to provide a snap-fit engagement between the receptacle of base (210) and sensor portion (212). It will be appreciated that sensor portion (212) may be fixedly coupled to base (210) in any other suitable manner, such as via adhesive and/or one or more fastener(s), etc.

As also mentioned above, base (210) partially defines a mounting assembly that is configured to attach to the head (H) of the patient (P). To that end, patient tracking assembly (200) further includes a pair of nose pads (220) having respective nose-gripping surfaces (222). Nose pads (220) are connected to opposing sides of base (210) via corresponding pad arms (230). As described in greater detail below, nose-gripping surfaces (222) may be sized and configured to cooperate with each other to pinch a portion of a nose, such as a nasal bridge, of the patient (P) to thereby fix base (210) to the head (H) of the patient (P). In the example shown, nose-gripping surfaces (222) are each generally hemispherical to maximize contact between nose-gripping surfaces (222) and the portion of the nose to be gripped. However, the particular configuration of each nose-gripping surface (222) may vary depending on the particular anatomy of the patient (P), such as the unique structure of the nasal bridge of the patient (P), as described in greater detail below. In this regard, at least a portion of each nose-gripping surface (222) may be sized and shaped to complement a corresponding unique structural feature of the nasal bridge of the patient (P). Base (210) may be sized and configured to rest against a portion of a nose and/or brow of the patient (P) when the nose of the patient (P) is pinched between nose-gripping surfaces (222). In the example shown, base (210) is generally square-shaped. However, the particular configuration of base (210) may vary as described in greater detail below.

In any event, base (210), nose pads (220), and pad arms (230) may each be substantially rigid and may be fixedly coupled to each other to inhibit movement of nose-gripping surfaces (222) relative to each other and/or relative to base (210). In some versions, base (210), nose pads (220), and pad arms (230) may be integrally formed together with each other as a single unitary (e.g., monolithic) piece. For example, base (210), nose pads (220), and pad arms (230) may be 3D-printed with each other as a single monolithic piece. In this manner, base (210), nose pads (220), and pad arms (230) may collectively define the mounting portion of patient tracking assembly (200) for securely mounting patient tracking assembly (200) to the nose of patient (P). While the term “pads” is used in the present designation of nose pads (220), the term “pad” and “pads” should not be read as implying that nose pads (220) are deformable in any way. In some versions, nose pads (220) are completely rigid and non-deformable. Moreover, the entirety of patient tracking assembly (200) (except for cable (214), in versions where cable (214) is used)) may be completely rigid and non-deformable. Nevertheless, nose-gripping surfaces (222) of nose pads (220) may have features that promote friction with skin of the face of the patient (P) or otherwise promote gripping of patient tracking assembly (200) on the face of the patient (P). Such features may include knurling, grip, a high-friction surface coating, or a thin layer of elastomer, etc. Alternatively, any other suitable kind(s) of features may be used to promote friction with skin of the face of the patient (P) or otherwise promote gripping of patient tracking assembly (200) on the face of the patient (P). In versions that provide a thin layer of elastomer at nose-gripping surfaces (222), such elastomer may only deform to a negligible degree (i.e., without meaningfully impacting the function of patient tracking assembly (200) providing accurate signals indicating the real-time position of the head (H) of the patient (P) in relation to the preoperative image(s)), such that the thin layer of elastomer does not meaningfully impact the overall rigidity of patient tracking assembly (200).

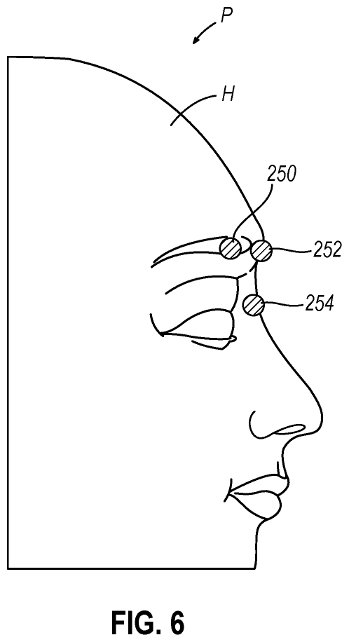

Referring now to FIGS. 6-8, at least the mounting portion of patient tracking assembly (200) may be custom manufactured for securely mounting patient tracking assembly (200) to the nose of a particular preselected patient (P). Prior to performing a surgical (e.g., ENT) procedure using image guidance, an operator (e.g., a physician) may acquire one or more preoperative images (e.g., scans) of the head (H) of the preselected patient (P), using any suitable imaging modality, such as computerized tomography (CT), cone beam computed tomography (CBCT), or magnetic resonance imaging (MRI). The image(s) may be used for planning the procedure beforehand, as well as for tracking the position of navigation guidewire (130) (or other instrument having a sensor compatible with IGS navigation system (100)) within the head (H) of the patient (P) during the procedure.

In some scenarios where the operator is performing an ENT procedure on the patient (P), or is part of a team performing an ENT procedure on the patient (P), the preoperative image(s) may have been acquired by different personnel, at a different time and/or at a different place, such that it is not necessary for the same person or team performing the ENT procedure to be the same person or team generating the preoperative image(s). Moreover, the preoperative image(s) need not necessarily have been acquired for the purpose of customized manufacturing of patient tracking assembly (200). The process of customized manufacturing of patient tracking assembly (200) may be carried out using preoperative image(s) that were originally acquired for some other purpose(s).

The anatomical image(s) may also be used for deriving instructions for manufacturing a customized mounting portion for patient tracking assembly (200) that is shaped to securely fit over the nose of the particular patient (P). In other words, the mounting portion of patient tracking assembly (200) may be manufactured to securely fit over the nose of the particular patient (P), but generally not the nose of any other patient. To that end, a process of customized manufacturing of patient tracking assembly (200) may utilize certain anatomical landmarks (250, 252, 254) on the face of the patient (P) as indexing points for the customized manufacturing process. In the present example, such anatomical landmarks (250, 252, 254) include the supraorbital foramen (250) on each side of the face of the patient (P), the glabella (252) on the face of the patient (P), and the radix (254) on the face of the patient (P).

It will be appreciated that the left and right supraorbital foramen (250), the glabella (252), and the radix (254) of the patient (P) may collectively define at least a portion of the nasal bridge of the patient (P). More particularly, the nasal bridge may be at least partially defined between the glabella (252) and the radix (254) along a first (e.g., generally vertical) axis, and between the left and right supraorbital foramen (250) along a second (e.g., lateral) axis. The nasal bridge of the patient (P) may include a relatively thin layer of skin above the underlying nasal bones, at least by comparison to the thicknesses of layers of skin elsewhere on the face of the patient (P), such that the nasal bridge may have minimal or no flexibility or pliability and may therefore be subject to minimal or no physical changes over a predetermined period of time (e.g., the period of time between acquisition of the anatomical image(s) and completion of the surgical procedure). Similarly, the nasal bridge of the patient (P) may exhibit minimal or no depression or other deformation when a predetermined pressing force is applied thereto (e.g., by nose pads (220)), and/or may experience minimal or no swelling during performance of an ENT procedure. Thus, the nasal bridge of the patient (P) may provide a consistently reliable anchoring location for supporting the mounting portion of patient tracking assembly (200) in a consistent, fixed manner relative to the head (H) of the patient (P) during the process of registering the head (H) of the patient (P) with IGS navigation system (100) at the beginning of an ENT procedure; and throughout the duration of an ENT procedure while IGS navigation system (100) is being used.

It will also be appreciated that the specific structure (e.g., size and shape) of the nasal bridge may be unique to the particular patient (P), which may be at least partially attributable to the specific structure of the underlying nasal/facial bones. Thus, the desired configurations of each nose-gripping surface (222) and/or of base (210) for achieving a secure fit of the mounting portion of patient tracking assembly (200) over the nose may vary based on the particular patient (P). For example, the particular sizes and shapes (e.g., surface contours) of each nose-gripping surface (222) and/or of base (210), as well as the gap between nose-gripping surfaces (222), for achieving such a secure fit may vary based on the particular patient (P). In this regard, it may be desirable for at least a portion of each nose-gripping surface (222) to be sized and shaped to complement a corresponding unique structural feature of the nasal bridge of patient (P) that the respective nose-gripping surface (222) lodges against when the mounting portion of patient tracking assembly (200) is fitted over the nose of the patient (P). Anatomical landmarks (250, 252, 254) may be representative of the unique structure of the nasal bridge of the particular patient (P); and may therefore be used to derive instructions for manufacturing a customized mounting portion for patient tracking assembly (200) (e.g., having the desired configurations of each nose-gripping surface (222) and/or of base (210)) that may securely fit over the nose of the particular patient (P).

In this regard, anatomical landmarks (250, 252, 254) may be identified in the preoperative image(s) and then be provided to a processor, such as processor (110), which may be configured to derive one or more instruction files for manufacturing a customized mounting portion for patient tracking assembly (200) based on scans (250, 252, 254). In some versions, image processing software is used to automatically identify the locations of anatomical landmarks (250, 252, 254) in the preoperative image(s). In some other versions, an operator observes the preoperative image(s) and uses a user input feature (e.g., mouse, touchscreen, etc.) to manually identify the locations of anatomical landmarks (250, 252, 254) in the preoperative image(s). As another variation, image processing software may initially automatically identify the locations of anatomical landmarks (250, 252, 254) in the preoperative image(s); and a user may then manually confirm or correct those automatically identified locations in the preoperative image(s).

In some versions where image processing software is used to automatically identify the locations of anatomical landmarks (250, 252, 254) in the preoperative image(s), fiducials may be used during the image acquisition process to assist in identifying the locations of anatomical landmarks (250, 252, 254). For instance, a user may place markers (e.g., stickers, marking elements, etc.) on the face of the patient (P) at the location of each anatomical landmark (250, 252, 254); and such markers may be visible or otherwise detectable in the preoperative image(s) to thereby facilitate subsequent identification. As another example, lasers or other forms of light may be projected toward the face of the patient (P), providing discretely illuminated regions at the location of each anatomical landmark (250, 252, 254). Such discretely illuminated regions may be visible or otherwise detectable in the preoperative image(s) to thereby facilitate subsequent identification. The foregoing examples are provided in a context where the preoperative image(s) is/are obtained for at least the purpose of facilitating manufacture of patient tracking assembly (200). However, as noted above, the customized manufacturing of patient tracking assembly (200) may be carried out using preoperative image(s) that were obtained for other purposes.

Regardless of how anatomical landmarks (250, 252, 254) have been identified in the preoperative image(s), the corresponding manufacturing instruction files may include the desired configurations of each nose-gripping surface (222) and/or of base (210) for securely fitting the mounting portion over the nose of the particular patient (P), such as configurations of nose-gripping surfaces (222) that complement the corresponding unique structural features (250, 252, 254) of the nasal bridge of patient (P). Processor (110) may be further configured to transmit such instruction files to an additive manufacturing device, such as a three-dimensional (3D) printer (not shown) that is configured to execute the instruction files to thereby produce the customized mounting portion for patient tracking assembly (200). In some versions, the 3D printer may be located at the surgical facility in which system (100) is situated, such as in the same room. In other versions, the 3D printer may be located at a remote production site separate from the surgical facility in which system (100) is situated. In such cases, the processor used to receive preoperative image(s) including anatomical landmarks (250, 252, 254), derive the manufacturing instruction files, and transmit the instruction files to the 3D printer may be separate from processor (110). In alternative versions, the customized mounting portion for patient tracking assembly (200) may be produced using any other suitable technique, such as techniques for producing molds for dental prostheses.

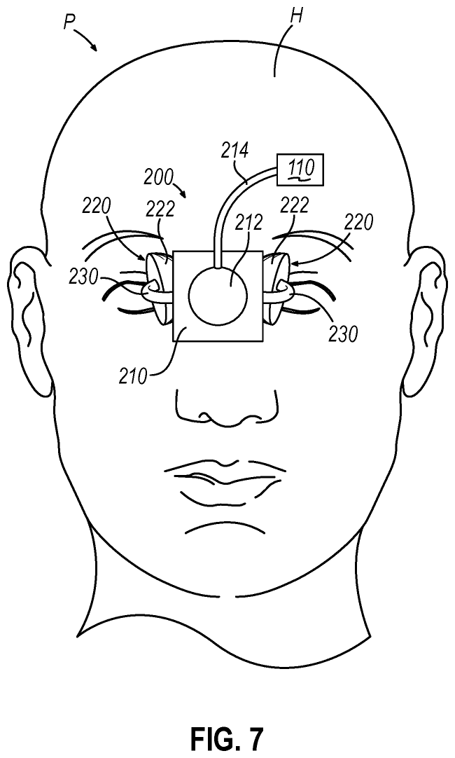

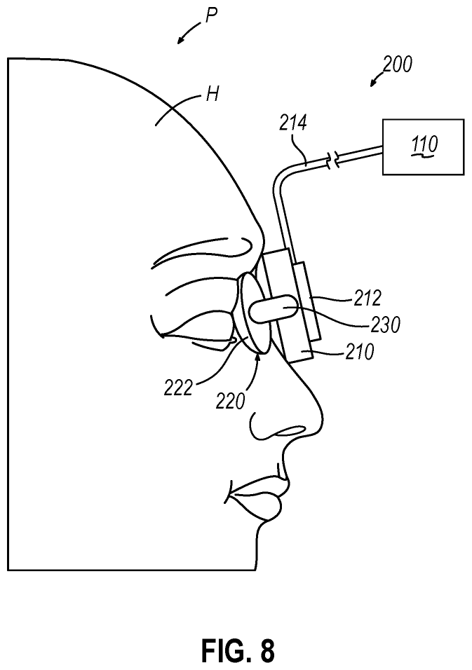

As shown in FIGS. 7 and 8, the customized mounting portion may be fixedly coupled with sensor portion (212) of patient tracking assembly (200), such as by snapping sensor portion (212) into the receptacle of base (210). The fully assembled patient tracking assembly (200) may then be fitted over the nose of the particular patient (P) such that sensor portion (212) may be fixed relative to the head (H) of the particular patient (P) at a known location relative to the anatomical landmarks (250, 252, 254). More particularly, the customized mounting portion of patient tracking assembly (200) may be securely supported on the nasal bridge of the particular patient (P). In the example shown, nose-gripping surfaces (222) of nose pads (220) pinch the nasal bridge of the patient (P) between the glabella (252) and the radix (254) of the patient (P) while base (210) rests against the glabella (252) and a portion of the nose at or near the rhinion. Due to the customized configurations of nose-gripping surfaces (222) of nose pads (220), the relatively thin layer of skin of the nasal bridge, and the rigidity of patient tracking assembly (200), nose-gripping surfaces (222) may securely grip the underlying nasal bone in a consistent and reliable manner before and during a surgical (e.g., ENT) procedure. Thus, the customized mounting portion of patient tracking assembly (200) may enable automatic registration of the anatomical image(s) to IGS navigation system (100) prior to the surgical procedure, thereby eliminating the need for manual registration by the physician during the surgical procedure. In other words, patient tracking assembly (200) may obviate the need to use a registration instrument like calibration instrument (134) as described above with reference to FIGS. 2-3. Furthermore, the accurate fixation of the customized mounting portion of patient tracking assembly (200) to the nose may reduce or eliminate the need to repeat the registration, for example, when the patient's head (H) moves.

In one example, a method of producing patient tracking assembly (200) begins with an imaging step at which a processor, such as processor (110), acquires one or more anatomical image(s) of the patient's head (H), such as CT, CBCT, or MRI image(s), including various predetermined anatomical landmarks on the face of the patient (P), such as left and right supraorbital foramen (250), the glabella (252), and the radix (254). The method may then proceed with an instruction deriving step, at which the processor analyzes the image(s) to identify in the image(s) the unique structure of the nasal bridge of the particular patient (P) and derives, based on the identified structure of the nasal bridge, one or more instruction files that comprises machine instructions for producing the mounting portion of patient tracking assembly (200). The method may then proceed with a production step, at which the processor sends the instruction files to a 3D printer that produces at least the mounting portion of patient tracking assembly (200). The method may then proceed with an assembly step, at which sensor portion (212) is fixedly attached to the mounting portion of patient tracking assembly (200). It will be appreciated that any one or more steps of the above method of producing patient tracking assembly (200) may be performed at the surgical facility in which system (100) is situated (e.g., by a physician) and/or at a remote production site separate from the surgical facility in which system (100) is situated (e.g., by a manufacturing technician).

In another example, a method of performing a surgical (e.g., ENT) procedure begins with an imaging step at which a processor, such as processor (110), acquires one or more anatomical image(s) of the patient's head (H), such as CT, CBCT, or MRI image(s). In some versions, this may be the same imaging step as that described above with respect to the method of producing patient tracking assembly (200). In such cases, the method of performing the surgical procedure may further include the instruction deriving, production, and/or assembly steps described above with respect to the method of producing patient tracking assembly (200). In any event, the method of performing the surgical procedure may then proceed with a fitting step, at which patient tracking assembly (200) is fitted over the nose of patient (P), such as with nose-gripping surfaces (222) of nose pads (220) pinching the nasal bridge of patient (P). The method may then proceed with a registration step, at which processor (110) registers the one or more anatomical image(s) with the coordinate system of IGS navigation system (100), such as by identifying the nasal bridge in the anatomical image(s) and measuring the position of sensor portion (212) in the coordinate system of IGS navigation system (100). It will be appreciated that after the registration step, the position of sensor portion (212) is anchored to the nasal bridge in the coordinate system and thus also in the anatomical image(s) during the procedure, even when the patient's head (H) moves. The method may then proceed with a surgical tool insertion step, at which the physician inserts a surgical tool, such as navigation guidewire (130), into the body of the patient (P), such as into an anatomical passageway within the patient's head (H) to perform a surgical intervention (e.g., balloon sinuplasty). It will be appreciated that the sensor of navigation guidewire (130) allows the physician to track the position of a distal end of guidewire (130) using IGS navigation system (100). In this regard, the method may proceed to a navigation step, at which the physician navigates the distal end of guidewire (130) to a target location (e.g., nasal sinus) using the sensor of guidewire (130), whose measured position may be overlaid on the anatomical image(s) displayed by display screen (114).

In some scenarios, by continuously tracking the position of sensor portion (212) relative to the sensor of guidewire (130), processor (110) may dynamically maintain the registration of the preoperative image(s) and the real-time position of the head (H) of the patient (P) with IGS navigation system (100) even when the patient (P) moves his/her head (H). Furthermore, processor (110) may continuously display the real-time position of guidewire (130) on the anatomical image(s) displayed by display screen (114), based on signals from the position sensor in guidewire (130) and the position sensor in patient tracking assembly (200), thus assisting the physician with conducting the surgical procedure.

In addition to the foregoing, patient tracking assembly (200) may be configured and operable in accordance with at least some of the teachings of U.S. Pat. No. 10,631,935, entitled “Head Registration Using a Personalized Gripper,” issued on Apr. 28, 2020, the disclosure of which is incorporated by reference herein, in its entirety.

### IV. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

An apparatus comprising: (a) a mounting portion configured to fit over a nose of a preselected patient, wherein the mounting portion includes: (i) a base, and (ii) a pair of rigid nose pads fixedly coupled to the base, wherein each nose pad of the pair of nose pads includes a respective nose-gripping surface, wherein the nose-gripping surfaces of the nose pads are configured to engage the nose of the preselected patient at respective predetermined locations along the nose, wherein each nose-gripping surface is sized and shaped to complement a corresponding unique structural feature of the nose of the preselected patient at the predetermined location; and (b) a sensor portion fixedly attached to the mounting portion, wherein the sensor portion includes a first sensor configured to generate a first signal corresponding to a position of the first sensor in three-dimensional space.

### EXAMPLE 2

The apparatus of Example 1, wherein the mounting portion further includes a pair of pad arms, wherein each nose pad of the pair of nose pads is coupled to the base via a corresponding pad arm of the pair of pad arms.

### EXAMPLE 3

The apparatus of any of Examples 1 through 2, wherein the sensor portion is fixedly attached to the mounting portion via a snap-fit engagement between the sensor portion and the mounting portion.

### EXAMPLE 4

The apparatus of Example 3, wherein the base of the mounting portion includes a receptacle for at least partially receiving the sensor portion to provide the snap-fit engagement between the sensor portion and the mounting portion.

### EXAMPLE 5

The apparatus of any of Examples 1 through 4, wherein the predetermined location is between a glabella of the preselected patient and a radix of the preselected patient along a first axis.

### EXAMPLE 6

The apparatus of Example 5, wherein the predetermined location is between a left supraorbital foramen of the preselected patient and a right supraorbital foramen of the preselected patient along a second axis.

### EXAMPLE 7

The apparatus of any of Examples 1 through 6, wherein the base and the pair of nose pads are integrally formed together with each other as a unitary piece.

### EXAMPLE 8

The apparatus of any of Examples 1 through 7, wherein the unique structural feature includes a portion of a nasal bridge of the preselected patient.

### EXAMPLE 9

The apparatus of Example 8, wherein the unique structural feature includes at least one nasal bone of the nasal bridge of the preselected patient.

### EXAMPLE 10

A system comprising: (a) the apparatus of any of Examples 1 through 9; (b) at least one magnetic field generator operable to generate a magnetic field around at least the nose of the preselected patient; and (c) a processor, wherein the processor is configured to drive the at least one magnetic field generator to generate the magnetic field, wherein the processor is configured to receive the first signal generated by the first sensor for tracking the position of the first sensor in three-dimensional space.

### EXAMPLE 11

The system of Example 10, further comprising a surgical tool, wherein the surgical tool includes a second sensor configured to generate a second signal corresponding to a position of the second sensor in three-dimensional space.

### EXAMPLE 12

The system of Example 11, wherein the processor is configured to receive the second signal generated by the second sensor for tracking the position of the second sensor in three-dimensional space.

### EXAMPLE 13

The system of any of Examples 10 through 12, wherein the processor is configured to acquire an anatomical image of the nose of the preselected patient.

### EXAMPLE 14

The system of Example 13, wherein the processor is configured to derive an instruction file for manufacturing the mounting portion of the apparatus based on the acquired anatomical image of the nose of the preselected patient.

### EXAMPLE 15

The system of Example 14, further comprising a 3D printer, wherein the 3D printer is configured to receive the instruction file from the processor, wherein the 3D printer is configured to produce the mounting portion of the apparatus based on the instruction file.

### EXAMPLE 16

A method of producing a patient tracking assembly, the method comprising: (a) acquiring an anatomical image of a nose of a preselected patient, wherein the anatomical image captures at least one unique structural feature of the nose of the preselected patient at a predetermined location along the nose; (b) deriving an instruction file based on the anatomical image for producing a mounting portion of the patient tracking assembly having at least one nose-gripping surface sized and shaped to complement the at least one unique structural feature; and (c) transmitting the instruction file to a 3D printer for producing the mounting portion.

### EXAMPLE 17

The method of Example 16, further comprising producing the mounting portion via the 3D printer.

### EXAMPLE 18

The method of Example 17, further comprising fixedly attaching a sensor portion of the patient tracking assembly to the mounting portion, wherein the sensor portion includes a sensor configured to generate a signal corresponding to a position of the sensor in three-dimensional space.

### EXAMPLE 19

A method of performing a surgical procedure using a patient tracking assembly including (i) a mounting portion, and (ii) a sensor portion having a first sensor configured to generate a first signal corresponding to a position of the first sensor in three-dimensional space, the method comprising: (a) acquiring an anatomical image of a nose of a preselected patient; (b) fitting a patient tracking assembly over the nose of the preselected patient at a predetermined location along the nose, wherein the mounting portion has at least one nose-gripping surface sized and shaped to complement at least one unique structural feature of the nose of the preselected patient at the predetermined location; (c) registering the anatomical image with an image-guided surgery (IGS) navigation system via a first sensor of the sensor portion; and (d) navigating a surgical tool along an anatomical passageway of the patient, wherein the surgical tool includes a second sensor configured to generate a second signal corresponding to a position of the second sensor in three-dimensional space.

### EXAMPLE 20

The method of Example 19, further comprising performing a surgical intervention within the anatomical passageway of the patient via the surgical tool.

### V. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/we claim:

1. An apparatus comprising:
(a) a mounting portion configured to fit over a nose of a preselected patient, wherein the mounting portion includes:
(i) a base, and
(ii) a pair of rigid nose pads fixedly coupled to the base, wherein each nose pad of the pair of nose pads includes a respective nose-gripping surface, wherein the nose-gripping surfaces of the nose pads are configured to engage the nose of the preselected patient at respective predetermined locations along the nose, wherein each nose-gripping surface is sized and shaped to complement a corresponding unique structural feature of the nose of the preselected patient at the predetermined location; and
(b) a sensor portion fixedly attached to the mounting portion, wherein the sensor portion includes a first sensor configured to generate a first signal corresponding to a position of the first sensor in three-dimensional space.

2. The apparatus of claim 1, wherein the mounting portion further includes a pair of pad arms, wherein each nose pad of the pair of nose pads is coupled to the base via a corresponding pad arm of the pair of pad arms.

3. The apparatus of claim 1, wherein the sensor portion is fixedly attached to the mounting portion via a snap-fit engagement between the sensor portion and the mounting portion.

4. The apparatus of claim 3, wherein the base of the mounting portion includes a receptacle for at least partially receiving the sensor portion to provide the snap-fit engagement between the sensor portion and the mounting portion.

5. The apparatus of claim 1, wherein the predetermined location is between a glabella of the preselected patient and a radix of the preselected patient along a first axis.

6. The apparatus of claim 5, wherein the predetermined location is between a left supraorbital foramen of the preselected patient and a right supraorbital foramen of the preselected patient along a second axis.

7. The apparatus of claim 1, wherein the base and the pair of nose pads are integrally formed together with each other as a unitary piece.

8. The apparatus of claim 1, wherein the unique structural feature includes a portion of a nasal bridge of the preselected patient.

9. The apparatus of claim 8, wherein the unique structural feature includes at least one nasal bone of the nasal bridge of the preselected patient.

10. A system comprising:
(a) the apparatus of claim 1;
(b) at least one magnetic field generator operable to generate a magnetic field around at least the nose of the preselected patient; and
(c) a processor, wherein the processor is configured to drive the at least one magnetic field generator to generate the magnetic field, wherein the processor is configured to receive the first signal generated by the first sensor for tracking the position of the first sensor in three-dimensional space.

11. The system of claim 10, further comprising a surgical tool, wherein the surgical tool includes a second sensor configured to generate a second signal corresponding to a position of the second sensor in three-dimensional space.

12. The system of claim 11, wherein the processor is configured to receive the second signal generated by the second sensor for tracking the position of the second sensor in three-dimensional space.

13. The system of claim 10, wherein the processor is configured to acquire an anatomical image of the nose of the preselected patient.

14. The system of claim 13, wherein the processor is configured to derive an instruction file for manufacturing the mounting portion of the apparatus based on the acquired anatomical image of the nose of the preselected patient.

15. The system of claim 14, further comprising a 3D printer, wherein the 3D printer is configured to receive the instruction file from the processor, wherein the 3D printer is configured to produce the mounting portion of the apparatus based on the instruction file.

16. A method of producing a patient tracking assembly, the method comprising:
(a) acquiring an anatomical image of a nose of a preselected patient, wherein the anatomical image captures at least one unique structural feature of the nose of the preselected patient at a predetermined location along the nose;
(b) deriving an instruction file based on the anatomical image for producing a mounting portion of the patient tracking assembly having at least one nose-gripping surface sized and shaped to complement the at least one unique structural feature; and
(c) transmitting the instruction file to a 3D printer for producing the mounting portion.

17. The method of claim 16, further comprising producing the mounting portion via the 3D printer.

18. The method of claim 17, further comprising fixedly attaching a sensor portion of the patient tracking assembly to the mounting portion, wherein the sensor portion includes a sensor configured to generate a signal corresponding to a position of the sensor in three-dimensional space.

19. A method of performing a surgical procedure using a patient tracking assembly including (i) a mounting portion, and (ii) a sensor portion having a first sensor configured to generate a first signal corresponding to a position of the first sensor in three-dimensional space, the method comprising:
(a) acquiring an anatomical image of a nose of a preselected patient;
(b) fitting a patient tracking assembly over the nose of the preselected patient at a predetermined location along the nose, wherein the mounting portion has at least one nose-gripping surface sized and shaped to complement at least one unique structural feature of the nose of the preselected patient at the predetermined location;
(c) registering the anatomical image with an image-guided surgery (IGS) navigation system via a first sensor of the sensor portion; and
(d) navigating a surgical tool along an anatomical passageway of the patient, wherein the surgical tool includes a second sensor configured to generate a second signal corresponding to a position of the second sensor in three-dimensional space.

20. The method of claim 19, further comprising performing a surgical intervention within the anatomical passageway of the patient via the surgical tool.
